(19) United States
(12) Patent Application Publication
(10) Pub. No.: US 2022/0370450 A1
(43) Pub. Date: Nov. 24, 2022

(54) CONTROLLED-RELEASE COMPOSITION FOR ORAL ADMINISTRATION COMPRISING COMPLEX OF ALPHA ADRENERGIC BLOCKER COMPOUND AND CLAY MINERAL

(71) Applicant: Korea Institute Of Geoscience And Mineral Resources, Daejeon (KR)

(72) Inventors: Il-Mo KANG, Seoul (KR); Dae-Duk KIM, Seoul (KR); Ju-Hwan PARK, Seoul (KR); Ki-Min ROH, Daejeon (KR); Jangik Ike LEE, Seoul (KR); Jaehwan KIM, Pohang-si (KR); Sung Man SEO, Pohang-si (KR); Su Young JUNG, Seoul (KR); Min-Jun BAEK, Seoul (KR); Hyun-Jong SHIN, Seoul (KR)

(73) Assignee: Korea Institute Of Geoscience And Mineral Resources, Daejeon (KR)

(21) Appl. No.: 17/620,577

(22) PCT Filed: May 29, 2020
(86) PCT No.: PCT/KR2020/006991

§ 371 Date: Dec. 17, 2021

(30) Foreign Application Priority Data
Jun. 20, 2019 (KR) 10-2019-0073749

(51) Int. Cl. A61K 31/517; A61K 9/00; A61K 9/14; A23L 33/10

(52) U.S. Cl. CPC A61K 31/517; A61K 9/0053; A61K 9/143; A23L 33/10

(57) ABSTRACT

Disclosed are a controlled-release composition for oral administration comprising a complex of a hydrophilic alpha adrenergic blocker compound or its salt; and a clay mineral, and a method for preparing the same. The composition of the present disclosure has an in vivo release rate controlled further than that of conventional alpha adrenergic blocker compounds, thereby preventing side effects caused by a rapid increase in plasma drug concentration.

### TECHNICAL FIELD

The present disclosure relates to a controlled-release composition for oral administration comprising a complex of an alpha adrenergic blocker compound and a clay mineral and a method for preparing same.

### BACKGROUND ART

Hypertension refers to a case where blood pressure is higher than a normal level, which usually refers to systolic blood pressure of 140 mmHg or higher or diastolic blood pressure of 90 mmHg or higher. The hypertension as a chronic disease causing all kinds of adult diseases, especially degenerative diseases of the circulatory system, is generated by various factors and uses various types of drugs according to the cause.

Currently, drugs used in the hypertension include an ACE inhibitors, a angiotensin receptor blocker, a beta blocker, a calcium blocker, a diuretic agent, an alpha adrenergic blocker, and the like (Korea Registration Patent No. 10-1943382, and Japan Publication Patent No. 2018-030894). Among them, as a primary therapeutic agent in the drug therapy, an ACE inhibitor, an angiotensin receptor blocker, a calcium blocker, a diuretic agent, and the like have been used, and the alpha adrenergic blocker has been used for a combination therapy additionally when the primary therapeutic agent is not effective.

One of the prostate diseases, prostate hypertrophy causes the lower bladder obstruction by two mechanisms, which are anatomical obstruction by an enlarged prostate and functional obstruction by neuromodulation dominating a prostate smooth muscle. Among them, the obstruction by the prostate smooth may expect the alleviation of symptoms by blocking the adrenergic nerves dominating the smooth muscle to relax the smooth muscle, and to this end, an alpha-1 adrenergic blocker may be used.

For treatment of hypertension and prostate hypertrophy, phenoxybenzamine, which is a non-selective alpha blocker, has been first used, but caused systemic side effects such as nasal congestion, orthostatic hypotension, arrhythmia, dizziness, headache, and the like and urological side effects reversal ejaculation and the like due to non-selectivity. Thereafter, selective alpha-1 receptor blockers, prazosin, terazosin, doxazosin, alfuzosin and tamsulosin have been developed and widely used. Particularly, middle-aged men have the prostate hypertrophy with hypertension, and in patients having both hypertension and prostate hypertrophy, the alpha-1 receptor blockers alleviate patient's urinary symptoms with a blood pressure drop effect.

The alpha-1 adrenergic blocker acts on an alpha-1 adrenergic receptor existing in the central and peripheral nerves to reduce the adrenergic stimulation acting on the blood vessels to lower the action of a vasoconstrictor. Accordingly, the peripheral blood vessels are expanded and the resistance in the vessels is lowered to lower the blood pressure. Since the prostate smooth muscle is known to be contracted by adrenergic stimulation, when the alpha-1 adrenergic blocker is used, the tension of the prostate and bladder is reduced to alleviate the symptoms of prostate hypertrophy.

A hypertension therapeutic agent is intended to keep constantly blood pressure, and the Food and Drug Administration (FDA) and the like have recommended that in order to prevent side effects such as postural hypotension or ischemic attack and minimize the range of fluctuation of blood pressure, a plasma concentration immediately before the next administration maintains a minimum effective plasma concentration, and a ratio of lowest plasma concentration/highest plasma concentration (trough/peak) of the blood pressure is ½ or higher. In other words, the hypertension therapeutic agent should exhibit the equal drug effect because the plasma concentration of the drug is constantly maintained for a long time within the effective plasma concentration range. In addition, the hypertension therapeutic agent should be taken for a long period of time, and in order to increase the convenience and drug compliance of the patient, it is preferred that the hypertension therapeutic agent is administered in a formulation once a day, and to this end, it is necessary to maintain the effective plasma concentration for 24 hours.

One of the alpha-1 adrenergic blockers, doxazosin was initially formulated as a rapid release tablet containing 2 mg or 4 mg of doxazosin mesylate. However, the rapid release tablet causes orthostatic hypotension due to a sudden increase in plasma concentration when first administered in 4 to 16 mg of a maintenance dose. In order to prevent this problem, the dose of the rapid release tablet starts at 1 mg once a day to 1 day and gradually increases to a maintenance dose of 4 to 16 mg for a few weeks.

Prazosin was initially formulated as a rapid release tablet containing prazosin hydrochloride. However, since there is a risk of orthostatic hypotension and fainting, the initial dose starts at 0.5 mg 2 to 3 times a day and gradually increases to up to 20 mg a day for few weeks.

Therefore, in order to reduce the inconvenience, sustained-release preparations have been studied, and as the sustained-release preparation, osmotic-controlled release preparations called Minipress-XL (prazosin HCl) and Cardura-XL have been developed in Pfizer Inc. The osmotic-controlled release preparations have an advantage of rapidly increasing the maintenance dose while reducing a lot of side effects by reducing a high initial plasma concentration due to the administration of the rapid release preparation. However, there is a disadvantage that the weight of one tablet of Cardura-XL containing 4 mg of doxazosin is 297 mg and an excipient of about 70 times of the drug capacity is included. In addition, there is a problem that a process of producing the osmotic-controlled release preparations is complicated and the production cost is high.

Therefore, the present inventors have conducted a study to improve the pharmacokinetics of existing alpha adrenergic blockers, and as a result, found that an alpha adrenergic blocker was continuously released from the complex at a low initial plasma concentration when orally administering the complex of a hydrophilic alpha adrenergic blocker compound or its salt; and a clay mineral, and then completed the present disclosure.

### DISCLOSURE

### Technical Problem

An object of the present disclosure is to provide a means capable of preventing a sudden increase in initial plasma drug concentration which may be caused when administering a hydrophilic alpha adrenergic blocker compound and increasing a blood drug half-life.

### Technical Solution

In order to achieve the object, the present disclosure provides a controlled-release composition for oral administration comprising a complex of a hydrophilic alpha adrenergic blocker compound or its salt; and a clay mineral.

Further, the present disclosure provides a method for preparing the controlled-release composition for oral administration.

### Advantageous Effects

According to the present disclosure, the controlled-release composition for oral administration is orally administered to a subject to control the release of the hydrophilic alpha adrenergic blocker compound in the composition, thereby stably and continuously maintaining the plasma drug concentration without a sudden change in plasma drug concentration.

### DESCRIPTION OF DRAWINGS

FIG. 1 shows a scanning electron microscopy photograph of a doxazosin compound itself.

FIG. 2 shows a scanning electron microscopy photograph of a doxazosin-bentonite complex prepared according to Example 2.

FIG. 3 shows a scanning electron microscopy photograph of a prazosin compound itself.

FIG. 4 shows a scanning electron microscopy photograph of a prazosin-bentonite complex prepared according to Example 4.

FIG. 5 illustrates X-ray diffraction analysis results of a doxazosin compound (Doxazosin), bentonite powder (Bentonite), a physical mixture of doxazosin and bentonite (Physical mixture), and a doxazosin-bentonite complex (Doxazosin-bentonite complex).

FIG. 6 illustrates X-ray diffraction analysis results of a prazosin compound (Prazosin), bentonite powder (Bentonite), a physical mixture of prazosin and bentonite (Physical mixture), and a prazosin-bentonite complex (Prazosin-bentonite complex).

FIG. 7 shows a graph of measuring drug release amounts over time on a doxazosin solution and a doxazosin-bentonite complex dispersion solution prepared according to Example 2.

FIG. 8 shows a graph of measuring drug release amounts over time on a prazosin solution, a prazosin-bentonite complex dispersion solution prepared according to Example 3, and a prazosin-bentonite complex dispersion solution prepared according to Example 4.

FIG. 9 illustrates a result of analyzing plasma drug concentrations over time after orally administering a doxazosin solution and a doxazosin-bentonite complex dispersion solution, respectively.

FIG. 10 illustrates a result of analyzing plasma drug concentrations over time after orally administering a prazosin solution and a prazosin-bentonite complex dispersion solution, respectively.

### BEST MODE FOR THE INVENTION

The present disclosure provides a controlled-release composition for oral administration comprising a hydrophilic alpha adrenergic blocker compound or its salt; and a clay mineral, wherein the release of the hydrophilic alpha adrenergic blocker compound in the composition is adjusted.

The hydrophilic alpha adrenergic blocker compound may be prazosin, doxazosin, terazosin, alfuzosin or a pharmaceutically acceptable salt thereof.

A time to maximum plasma concentration (tmax) in the oral administration of the composition may be 1.5 to 13 times longer than a time to maximum plasma concentration (tmax) in the oral administration of the hydrophilic alpha adrenergic blocker compound aqueous solution.

The time to maximum plasma concentration (tmax) in the oral administration of the composition may be longer than the time to maximum plasma concentration (tmax) in the oral administration of the hydrophilic alpha adrenergic blocker compound aqueous solution.

A maximum plasma concentration (Cmax) in the oral administration of the composition may be 15% to 80% of a maximum plasma concentration (Cmax) in the oral administration of the hydrophilic alpha adrenergic blocker compound aqueous solution.

The maximum plasma concentration (Cmax) in the oral administration of the composition may be lower than the maximum plasma concentration (Cmax) in the oral administration of the hydrophilic alpha adrenergic blocker compound aqueous solution.

An elution rate when adding the complex to an eluate of pH 1.2 may be slower than the elution rate when adding the hydrophilic alpha adrenergic blocker compound to the eluate of pH 1.2.

An elution rate when adding the complex to an eluate of pH 7.8 may be slower than the elution rate when adding the hydrophilic alpha adrenergic blocker compound to the eluate of pH 7.8.

The composition may be a pharmaceutical composition for preventing or treating hypertension or prostate hypertrophy.

The composition may be a food composition for preventing or alleviating hypertension or prostate hypertrophy.

Further, the present disclosure provides a method for preparing a controlled-release composition for oral administration comprising preparing a complex by mixing a drug aqueous solution prepared by dissolving an alpha adrenergic blocker compound or its pharmaceutically acceptable salt in an acidic aqueous solvent; and a clay mineral suspension to adsorb the compound onto the clay mineral.

The drug aqueous solution may have pH of more than 0 to less than 7.

### MODE FOR THE INVENTION

The present disclosure provides a controlled-release composition for oral administration comprising a complex of a hydrophilic alpha adrenergic blocker compound or its salt; and a clay mineral.

The present disclosure provides a method for preparing a controlled-release composition for oral administration comprising a complex of a hydrophilic alpha adrenergic blocker compound or its salt; and a clay mineral.

Hereinafter, embodiments of the present disclosure will be described with reference to the accompanying drawings. The present disclosure may have various modifications and various embodiments and specific embodiments will be illustrated in the drawings and described in detail in the specification. However, this does not limit the present disclosure to specific embodiments, and it should be understood that the present disclosure covers all the modifications, equivalents and replacements included within the idea and technical scope of the present disclosure.

Terms including as first, second, and the like may be used for describing various components, but the components are not limited by the terms. The terms are used only to discriminate one constituent element from another constituent element. For example, without departing from the scope of the invention, a first constituent element may be named as a second constituent element, and similarly a second constituent element may be named as a first constituent element.

Terms used in the present application are used only to describe specific embodiments, and are not intended to limit the present disclosure. Singular expressions used herein include plurals expressions unless they have definitely opposite meanings in the context. In the present application, it should be understood that term “comprising” or “having” indicates that a feature, a number, a step, an operation, a component, a part or a combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as ideal or excessively formal meanings unless otherwise defined in the present application.

Clay Mineral

The present disclosure relates to a controlled-release composition for oral administration comprising a complex of a hydrophilic alpha adrenergic blocker compound or its salt; and a clay mineral.

In general, a clay mineral has a layered structure, that is, a plate-like structure in which crystal units formed by combining silica sheets and alumina sheets are stacked, and in a clay mineral having interlayer expansibility among these clay minerals, since the binding force between the crystal units is weak without hydrogen bonds between the crystal units, moisture is introduced between the crystal units to be expanded. Therefore, it is possible to easily introduce even ions having relatively large sizes between the crystal units of the clay mineral having interlayer expansibility. Meanwhile, in the clay mineral having interlayer expansibility, tetrahedral Si having a tetravalent positive charge is isomorphic-substituted to Al or Fe having a trivalent positive charge or octahedral Al or Fe³⁺ having a trivalent positive charge is isomorphic-substituted to Mg or Fe²⁺ having a bivalent positive charge to generate a negative layer charge, but cations such as calcium ions (Ca²⁺), magnesium ions (Mg²⁺), sodium ion (Na⁺), potassium ions (K⁺), and the like are bound between the layers or on the surface to have entirely electrical neutrality. The clay mineral of the present disclosure is excellent in adsorption of a cationic substance, and such an adsorption pattern depends on an ambient pH.

The clay mineral of the present disclosure is a clay mineral which has a plate-like structure, specifically, interlayer expansibility, and may be used as a carrier by inserting an antibiotic into the clay mineral. The clay mineral of the present disclosure may be smectite-based minerals, for example, montmorillonite or bentonite, smectite, vermiculite, beidellite, nontronite, saponite, hectorite, and the like. For example, the clay mineral of the present disclosure may be bentonite containing 50 wt % or more of montmorillonite. Preferably, the clay mineral of the present disclosure is bentonite.

Hydrophilic Alpha Adrenergic Blocker Compound

The present disclosure relates to a controlled-release composition for oral administration comprising a complex (hereinafter, referred to as “complex of the present disclosure) of a hydrophilic alpha adrenergic blocker compound or its salt; and a clay mineral.

The alpha adrenergic blocker compound may comprise a drug compound that serves to act to an alpha receptor of adrenergic neurotransmitter receptors to block the action thereof. The hydrophilic alpha adrenergic blocker compound of the present disclosure or its salt may be prazosin, doxazosin, terazosin, alfuzosin or pharmaceutically acceptable salts thereof. For example, the aqueous hydrophilic alpha adrenergic blocker compound of the present disclosure or its salt may be prazosin hydrochloride, doxazosin mesylate, terazosin hydrochloride, alfuzosin hydrochloride, and the like, but is not limited thereto.

The prazosin and the prazosin hydrochloride may have chemical structures of the following Chemical Formulas 1 and 2, the doxazosin and the doxazosin mesylate may have chemical structures of the following Chemical Formulas 3 and 4, the terazosin and the terazosin hydrochloride may have chemical structures of the following Chemical Formulas 5 and 6, and the alfuzosin and the alfuzosin hydrochloride may have chemical structures of the following Chemical Formulas 7 and 8.

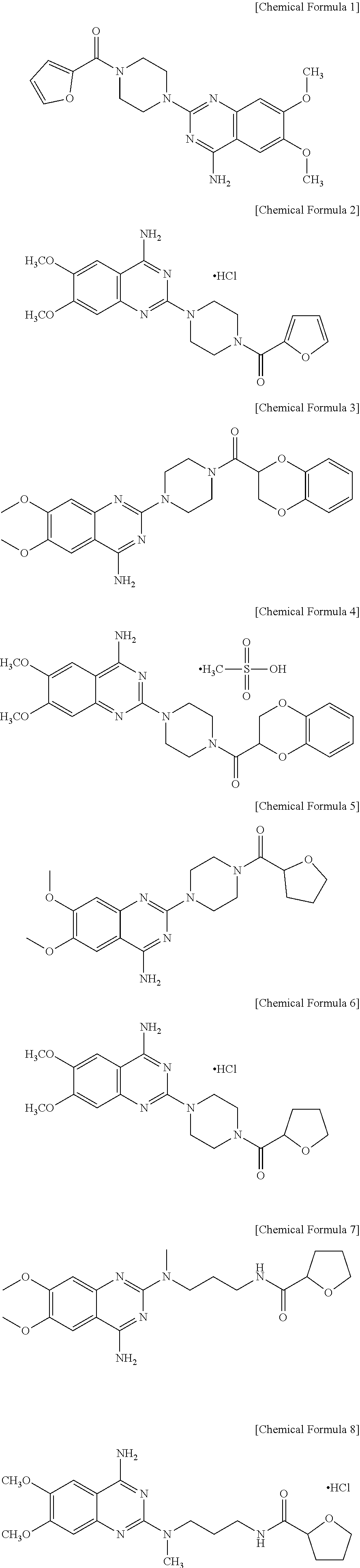

Complex of Hydrophilic Alpha Adrenergic Blocker Compound or its Salt; and Clay Mineral

In the complex of the present disclosure, the alpha adrenergic blocker compound may be ionic-bonded or physically adsorbed onto the clay mineral. For example, the alpha adrenergic blocker compound may be bound or adsorbed between crystal units or onto the surface of the clay mineral having anions in a cationic form to form a complex bound between the plate-like structures of the clay mineral.

In the complex of the present disclosure, while the clay mineral moves from the upper digestive tract to the lower digestive tract when the complex is attached to the intestinal wall, the hydrophilic alpha adrenergic blocker compound bound in the complex is eluted at a low elution rate and persistence. Therefore, in the complex of the present disclosure, the clay mineral contributes to maintaining a relatively constant plasma drug concentration through a drug release control as a carrier of the hydrophilic alpha adrenergic blocker compound. The clay mineral of the present disclosure is a layered clay mineral, the layered surface has a negative charge, and the alpha adrenergic blocker compound is adsorbed onto the clay mineral in an amorphous state. The complex may comprise the clay mineral and the hydrophilic alpha adrenergic blocker compound or its pharmaceutically acceptable salt in a weight ratio of 1:0.01 to 1. When the ratio of the hydrophilic alpha adrenergic blocker compound or its pharmaceutically acceptable salt is more than 1, there may be a problem that the amount of a drug compound lost in the process of preparing the complex is increased. When the ratio of the hydrophilic alpha adrenergic blocker compound or its pharmaceutically acceptable salt is less than 0.01, there may be a problem that the amount of the complex to be taken to administer the same drug amount is excessively increased.

When orally administering the hydrophilic alpha adrenergic blocker compound on the pharmacokinetics, a plasma drug concentration is initially rapidly increased and a maximum plasma concentration is also high, and as a result, it is difficult to expect the continuous action of the drug and there are side effects such as orthostatic hypotension and the like due to the sudden increase in plasma drug concentration.

However, in the case of the complex of the present disclosure, preferably, a drug-bentonite complex in which bentonite and the hydrophilic alpha adrenergic blocker compound or its salt are adsorbed, since bentonite is attached to the small intestinal epithelial cells due to an application capacity and then the hydrophilic alpha adrenergic blocker compound as the drug is released and absorbed, the complex can be absorbed in the entire small intestine area. Moreover, while bentonite is attached to the intestinal wall and moves to the lower digestive tract, the hydrophilic alpha adrenergic blocker compound which has been adsorbed in the complex is continuously released to maintain the continuous and stable plasma drug concentration in the body without a sudden change in the plasma drug concentration in the body.

Controlled-Release Composition for Oral Administration

The present disclosure relates to a controlled-release composition for oral administration comprising a complex of a hydrophilic alpha adrenergic blocker compound or its salt; and a clay mineral, and a method for preparing the same.

When the controlled-release composition for oral administration of the present disclosure is orally administered to a subject, the hydrophilic alpha adrenergic blocker compound is stably delivered to the lower digestive tract through the upper digestive tract while adsorbed onto the clay mineral to help so that the drug release is delayed and the drug is continuously released and absorbed. In addition, the controlled-release composition for oral administration of the present disclosure prevents the sudden absorption of the drug immediately after administration. In addition, since the controlled-release composition is well dispersed in water, the absorption is easier than the tablets and reproducibly occurs.

When the controlled-release composition for oral administration of the present disclosure is orally administered to rats, a time to maximum plasma concentration (tmax) may be 30 to 60 minutes, and the time to maximum plasma concentration (tmax) when orally administered to the human may be 1 to 10 minutes. In addition, the time to maximum plasma concentration (tmax) in the oral administration of the composition is longer than the time to maximum plasma concentration (tmax) in the oral administration of the hydrophilic alpha adrenergic blocker compound aqueous solution. Preferably, the time to maximum plasma concentration (tmax) in the oral administration of the composition is 1.5 to 13 times longer than the time to maximum plasma concentration (tmax) in the oral administration of the hydrophilic alpha adrenergic blocker compound aqueous solution. More preferably, a time to maximum plasma concentration (tmax) in the oral administration of a composition for oral administration comprising a doxazosin-clay mineral complex is 1.5 to 6 times, more preferably 2 to 4 times longer than the time to maximum plasma concentration (tmax) in the oral administration of a doxazosin solution. More preferably, a time to maximum plasma concentration (tmax) in the oral administration of a composition for oral administration comprising a prazosin-clay mineral complex is 6 to 13 times, more preferably 8 to 12 times longer than the time to maximum plasma concentration (tmax) in the oral administration of a prazosin solution.

In the oral administration of the controlled-release composition for oral administration comprising the complex of the hydrophilic alpha adrenergic blocker compound or its salt; and the clay mineral, a maximum plasma concentration (Cmax) may be 10 to 60 ng/ml. Preferably, in the oral administration of the composition for oral administration comprising the doxazosin-clay mineral complex, a maximum plasma concentration (Cmax) may be 10 to 35 ng/ml. Preferably, in the oral administration of the composition for oral administration comprising the prazosin-clay mineral complex, a maximum plasma concentration (Cmax) may be 33 to 60 ng/ml.

In the oral administration of the controlled-release composition for oral administration of the present disclosure comprising the complex of the hydrophilic alpha adrenergic blocker compound or its salt; and the clay mineral, the maximum plasma concentration (Cmax) is lower than the maximum plasma concentration (Cmax) in the oral administration of the hydrophilic alpha adrenergic blocker compound aqueous solution. Preferably, the maximum plasma concentration (Cmax) in the oral administration of the composition is 15% to 80%, preferably 20% to 60%, more preferably 25% to 45% of the maximum plasma concentration (Cmax) in the oral administration of the hydrophilic alpha adrenergic blocker compound aqueous solution. More preferably, in the oral administration of the composition for oral administration comprising the doxazosin-clay mineral complex, the maximum plasma concentration (Cmax) may be 15 to 40 ng/ml. More preferably, in the oral administration of the composition for oral administration comprising the prazosin-clay mineral complex, the maximum plasma concentration (Cmax) may be 30 to 55 ng/ml.

An elution rate when adding the complex of the hydrophilic alpha adrenergic blocker compound or its salt; and the clay mineral of the present disclosure to an eluate of pH 1.2 is slower than the elution rate when adding the hydrophilic alpha adrenergic blocker compound to the eluate of pH 1.2. In addition, the elution rate when adding the complex to an eluate of pH 7.8 is slower than the elution rate when adding the hydrophilic alpha adrenergic blocker compound to the eluate of pH 7.8. Therefore, the complex of the present disclosure has an elution rate slower than the hydrophilic alpha adrenergic blocker compound under both conditions of the upper digestive tract with a low pH and the lower digestive tract with a high pH.

In addition, in the oral administration of the controlled-release composition for oral administration of the present disclosure comprising the complex of the hydrophilic alpha adrenergic blocker compound or its salt; and the clay mineral, the time to maximum plasma concentration (tmax) is increased and the maximum plasma concentration (Cmax) is decreased as compared with the case of administering the compound itself due to the slow and continuous release of the drug, thereby preventing a sudden increase in plasma concentration. Therefore, the composition for oral administration of the present disclosure has an effect of reducing side effects such as orthostatic hypotension and the like due to a sudden increase in initial plasma concentration.

The composition of the present disclosure is a controlled-release composition. The modified- or controlled-release means that the plasma concentration of the drug after administering the drug is rapidly increased to an effective concentration, the plasma drug concentration is constantly maintained only for a desired time, an administration frequency is lower than those of general other preparations, a bioreaction is uniform, and side effects are small.

The composition of the present disclosure may be a pharmaceutical composition. Preferably, the composition of the present disclosure may be a pharmaceutical composition for preventing or treating hypertension or prostate hypertrophy. The pharmaceutical composition of the present disclosure may comprise 0.01 to 80 wt %, preferably 0.02 to 65 w % of the complex of the present disclosure. However, the amount may be increased or decreased according to the needs of a user, and may be appropriately increased or decreased depending on a situation, such as age, diet, nutrition condition, and disease progression. The content of the complex of the hydrophilic alpha adrenergic blocker compound or its pharmaceutically acceptable salt; and the clay mineral in the pharmaceutical composition of the present disclosure may be appropriately determined by those skilled in the art.

The pharmaceutical composition of the present disclosure can be administered orally and be used in forms of general pharmaceutical preparations. For example, the pharmaceutical composition of the present disclosure may be used in the form of preparations for oral administration, such as tablets, granules, capsules, suspensions, and the like, and these preparations may be prepared using general acceptable carriers, for example, in the case of orally-administered preparations, excipients, binders, disintegrants, lubricants, solubilizers, coloring agents, coating agents, suspensions, preservatives, or extenders. This is an example of preparation forms and additives that are applicable in the preparation of the pharmaceutical composition of the present disclosure, and the pharmaceutical composition of the present disclosure is not limited thereto.

The dose of the pharmaceutical composition of the present disclosure may be determined by experts according to various factors such as the condition, age, sex, and complications of patients, but may be generally administered in a dose of 0.1 mg to 10 g, preferably 10 mg to 5 g per 1 kg of adult. Further, the pharmaceutical composition per unit formulation is contained in a daily dose or ½, ⅓, or ¼ dose thereof, and may be administered 1 to 6 times a day, but is not limited thereto, and an attending physician may appropriately control the amount.

The composition of the present disclosure may be a food composition. Preferably, the composition of the present disclosure may be a food composition for prevention or alleviation of hypertension or prostate hypertrophy.

The controlled-release composition for oral administration of the present disclosure may include additives in addition to the complex of the present disclosure.

Complex powders of the present disclosure may be mixed directly with the additives, or may be mixed with the additives in the form encapsulated in soft capsules, for example, a tablet form. When the complex powders are encapsulated in hard capsules, in order to help in release and disintegration of the alpha adrenergic blocker compound, at least one selected from disintegrators, excipients, sustained release modifying agents, slip modifiers, and the like may be added in the hard capsules.

The additives may improve the physical properties of the complex powder in the human body and adjust the release rate of the alpha adrenergic blocker compound. For example, the additives may include one or more selected from polyethylene glycol, polyvinylpyrrolidone, polyoxyethylene sorbitan monooleate (product name: tween-80), poloxamer, poly-oxyethylene esters of 12-hydroxystearic acid (Solutol HS15), carbomer, sodium taurocholate, and the like.

In an exemplary embodiment, the additives may further include one or more selected from hydroxypropylmethylcellulose, Eudragit, lactose, and the like in order to further enhance the drug sustained-release ability of the complex.

In an exemplary embodiment, the oral pharmaceutical composition may comprise the additives in about 0.5 to 30 wt % based on the total weight.

Method for Preparing Controlled-Release Composition for Oral Administration of the Present Disclosure

The present disclosure relates to a method for preparing a controlled-release composition for oral administration comprising a complex of a hydrophilic alpha adrenergic blocker compound or its salt; and a clay mineral of the present disclosure. The preparing method may comprise preparing a complex by mixing a drug aqueous solution prepared by dissolving the alpha adrenergic blocker compound or its pharmaceutically acceptable salt in an acidic aqueous solvent; and a clay mineral suspension to adsorb the compound onto the clay mineral. At this time, a method of dissolving, dispersing, and mixing the compound and the clay mineral in the same solution may be used so that the hydrophilic alpha adrenergic blocker compound is adsorbed onto the layered clay mineral in an amorphous state. At this time, since the layered surface of the clay mineral (e.g., bentonite) has a negative charge, the adsorption may be more effectively performed when the drug dissolved in the solution has cations.

The method for preparing the controlled-release composition for oral administration may comprise a first step (S110) of preparing a drug aqueous solution by dissolving the alpha adrenergic blocker compound in an acidic aqueous solvent; a second step (S120) of preparing a clay mineral suspension by dispersing clay mineral powder in an aqueous solvent; and a third step (S130) of binding molecules of the alpha adrenergic blocker compound to the clay mineral powder by mixing the drug aqueous solution and the clay mineral suspension.

Further, the method for preparing the controlled-release composition for oral administration may comprise a first step (M110) of preparing a clay mineral suspension by dispersing clay mineral powder in an aqueous solvent; a second step (M120) of preparing a drug aqueous solution by dissolving the alpha adrenergic blocker compound in an acidic aqueous solvent; and a third step (M130) of binding molecules of the alpha adrenergic blocker compound to the clay mineral powder by mixing the clay mineral suspension and the drug aqueous solution.

At this time, one of hydrochloric acid, phosphoric acid, sulfuric acid, acetic acid, and formic acid may be selected to impart acid conditions to the reaction solution. The drug aqueous solution is preferably more than 0 to less than 7 of pH, more preferably pH 0.5 to 3, much more preferably pH 0.6 to 2.5, and even more preferably pH 0.6 to 1.6.

In the drug aqueous solution, the concentration of the alpha adrenergic blocker compound may be about 0.1 or more to 50 mg/mL.

In the step (S120, M110) of preparing the clay mineral suspension, the concentration of the clay mineral powder in the clay mineral suspension may be about 0.1 to 50 mg/mL.

In the third step (S130, M130), the mixed solution may be stirred using a stirrer to bind the molecules of the ionized alpha adrenergic blocker compound between the layers of the clay mineral powder after mixing the drug aqueous solution and the clay mineral suspension.

Meanwhile, since the amount of the alpha adrenergic blocker compound capable of binding to the clay mineral powder is limited, it is preferred to set a mixing ratio of the clay mineral powder and the alpha adrenergic blocker compound in the mixed solution, in consideration of the content of the drug compound contained in the drug-clay mineral complex powder, a ratio of the alpha adrenergic blocker compound which is not bound to the clay mineral powder but lost, and the like.

At this time, for sufficient adsorption, it is preferred that the complex contains the clay mineral and the hydrophilic alpha adrenergic blocker compound or its pharmaceutically acceptable salt in a weight ratio of 1:0.01 to 1. When reacting the excess drug (hydrophilic alpha adrenergic blocker compounds) in the clay mineral, the drug that is not adsorbed onto the clay mineral but lost is increased. On the other hand, when the amount of the clay mineral to the drug amount is increased, the entire adsorption rate may be increased, but as a result, the drug content of the complex is lowered and the dosage is increased to be burdened in administration.

In certain conditions, various methods may be used to obtain the complex in a dried form after forming the complex of the present disclosure. In addition, after the hydrophilic alpha adrenergic blocker compound is adsorbed onto the clay mineral, the non-bound hydrophilic alpha adrenergic blocker compound may be removed through centrifugation, supernatant removal, etc. Further, when the precipitate is lyophilized, the complex having a similar powder shape to the existing clay mineral may be obtained. Meanwhile, in order to separate the reaction solution and the complex, a process of passing only the solvent through the filter and repeatedly washing the non-passed complex precipitate with distilled water may be included to remove the non-adsorbed drug and the acidic solution without centrifugation. Alternatively, after completion of the adsorption reaction, a method of lyophilization by mitigating the strong acidic condition of the solution may be used. Specifically, after the reaction is performed at a low pH in which the drug may be efficiently adsorbed, the lyophilization is performed by increasing the pH of the reaction solution is increased, thereby reducing the likelihood of the mechanical corrosion and smoothly operating a lyophilizer.

Therefore, the method for preparing the controlled-release composition for oral administration of the present disclosure may further comprise a fourth step (S140, M140) of centrifuging the mixed solution of the drug aqueous solution and the clay mineral suspension in which the reaction is completed to remove a supernatant and drying the remaining complex powder.

Method for Preventing or Treating Hypertension or Prostate Hypertrophy

The present disclosure relates to a method for preventing or treating hypertension or prostate hypertrophy comprising orally administering a controlled-release composition for oral administration to a subject, the composition comprising a complex of a hydrophilic alpha adrenergic blocker compound or its salt; and a clay mineral. At this time, the composition may be a pharmaceutical composition for preventing or treating hypertension or prostate hypertrophy.

The subject to be administered with the controlled-release composition for oral administration of the present disclosure may be humans, mammals, or mammals except for humans, diagnosed as disease applied with the hydrophilic alpha adrenergic blocker compound or regarded as having a risk of the disease.

Method for preventing or alleviating hypertension or prostate hypertrophy

The present disclosure relates to a method for preventing or alleviating hypertension or prostate hypertrophy comprising orally administering a controlled-release composition for oral administration to a subject, the composition comprising a complex of a hydrophilic alpha adrenergic blocker compound or its salt; and a clay mineral. Preferably, the composition may be a food composition for preventing or alleviating hypertension or prostate hypertrophy.

The subject to be administered with the controlled-release composition for oral administration of the present disclosure may be humans, mammals, or mammals except for humans, diagnosed as disease applied with the hydrophilic alpha adrenergic blocker compound or regarded as having a risk of the disease.

Hereinafter, some Examples and Experimental Examples of the present disclosure will be described. However, the following Examples are just some embodiments of the present disclosure, and it should not be understood that the scope of the present disclosure is limited to the following Examples.

<Materials and Methods>

Bentonite was received and used by the Korea Institute of Geoscience and Mineral Resources. The experimental animals used in Experimental Example 5 were a Sprague Dawley Rats (SD rats). 8-week-old SD rats were purchased from ORIENT BIO Inc. and experimented in an experimental animal room at the first basement, 143, College of Medicine of Seoul National University. The breeding conditions of the experimental animals were as follows. Temperature and humidity range of 22±2° C., 50±5% (RH). ventilation count of 10 to 15 times/hour, lighting time and illuminance 12 hours lighting on (lighting: 08:00 to 20:00), illuminance: 150 to 300 Lux.

### Example 1

A certain amount of doxazosin compound was dissolved in distilled water at room temperature and then added with 20 ml of a 0.1 N hydrochloric acid solution to prepare a drug aqueous solution at an acidic condition of pH 1.2.

Subsequently, bentonite powder having the same mass as the doxazosin compound was suspended in distilled water to prepare a bentonite suspension. That is, doxazosin and bentonite were used at a weight ratio of 1:1.

Subsequently, the drug aqueous solution and the bentonite suspension were mixed and stirred for 1 hour to prepare a doxazosin-bentonite complex. At this time, the bentonite suspension was added to the drug aqueous solution in a continuous stirring state so as not to precipitate the bentonite powder.

Subsequently, the doxazosin-bentonite complex was centrifuged at 3,000 rpm for 10 minutes to be precipitated to remove the supernatant and the remaining pellets were rapidly cooled using liquid nitrogen and then lyophilized to evaporate the remaining solvent.

### Example 2

Except of preparing a bentonite dispersion using the mass of bentonite powder two times larger than the mass of the doxazosin compound, a doxazosin-bentonite complex was prepared in the same manner as Example 1. That is, doxazosin and bentonite were used at a weight ratio of 1:2.

### Example 3

Except for using a prazosin compound instead of the doxazosin compound to prepare a drug aqueous solution, a prazosin-bentonite complex was prepared in the same manner as Example 1. That is, prazosin and bentonite were used at a weight ratio of 1:1.

### Example 4

Except for using a prazosin compound instead of the doxazosin compound to prepare a drug aqueous solution and preparing a bentonite dispersion using the mass of bentonite powder two times larger than the mass of the prazosin compound, a prazosin-bentonite complex was prepared in the same manner as Example 1. That is, prazosin and bentonite were used at a weight ratio of 1:2.

### Example 5

Except for using a terazosin compound instead of the doxazosin compound to prepare a drug aqueous solution, a terazosin-bentonite complex was prepared in the same manner as Example 1. That is, terazosin and bentonite were used at a weight ratio of 1:1.

### Example 6

Except for using an alfuzosin compound instead of the doxazosin compound to prepare a drug aqueous solution, an terazosin-bentonite complex was prepared in the same manner as Example 1. That is, alfuzosin and bentonite were used at a weight ratio of 1:1.

### Comparative Example 1

A doxazosin compound and bentonite powder were added in a test tube at a weight ratio of 1:2 and mixed using a Vortexer for 30 minutes to prepare a physical mixture of doxazosin and bentonite.

### Comparative Example 2

A prazosin compound and bentonite powder were added in a test tube at a weight ratio of 1:2 and mixed using a Vortexer for 30 minutes to prepare a physical mixture of prazosin and bentonite.

Table 1 below illustrates the drug compounds used in Examples 1 to 6 and composition ratios of the drug compounds and the bentonite powder used when preparing the complex.

TABLE 1

| Example | Drug | Composition ratio of drug:bentonite |
|---|---|---|
| 1 | Doxazosin | 1:1 |
| 2 | Doxazosin | 1:2 |
| 3 | Prazosin | 1:1 |
| 4 | Prazosin | 1:2 |
| 5 | Terazosin | 1:1 |
| 6 | Alfuzosin | 1:2 |

### Experimental Example 1

With respect to the drug-bentonite complexes prepared in Examples 1 to 4, the adsorption rate of the drug and the drug content per unit weight were confirmed.

The adsorption rate of the drug was calculated by indirectly calculating the drug amount adsorbed onto bentonite like Equation 1 by excluding the drug amount remaining in the supernatant of the entire drug applied to the preparing process of the complex.

$$Adsorption rate ( \% ) = \frac{\begin{matrix}amount ( m g ) of entirely applied drug - \\ amount ( m g ) of drug remaining in supernatant\end{matrix}}{amount ( m g ) of entirely applied drug} \times 100 \qquad [ Equation 1 ]$$

On the other hand, a supernatant to be obtained after centrifugation was subjected to a syringe filter to prevent bentonite particles from affecting the analysis. The supernatant obtained after centrifugation was diluted with a mixed solution of acetonitrile and distilled water to measure the drug concentration with high performance liquid chromatography.

As a result, in the complexes obtained in Examples 1 to 4, the drug adsorption rate and the drug content per unit weight were shown in Table 2 below.

TABLE 2

| | | Drug content per complex |
|---|---|---|
| | Adsorption rate | unit weight (drug |
| Example | of drug(%) | (mg)/complex (g)) |
| 1 | 50.65 ± 2.97 | 336.2 |
| 2 | 97.20 ± 3.06 | 327.1 |
| 3 | 61.05 ± 0.30 | 378.8 |
| 4 | 79.23 ± 1.30 | 283.3 |

Referring to Table 2, when the weight ratio of the drug compound and the bentonite powder was 1:1 (Examples 1 and 3), the drug adsorption rate was lower than that when the weight ratio thereof was 1:2 (Examples 2 and 4), but the drug content per complex unit weight was higher therethan.

### Experimental Example 2

The form of the drug-bentonite complex was observed using a scanning electron microscope (SEM) to confirm the morphological properties of the prepared drug-bentonite complex. The SEM image was photographed by adsorbing the drug-bentonite complex onto a copper tape and performing platinum coating.

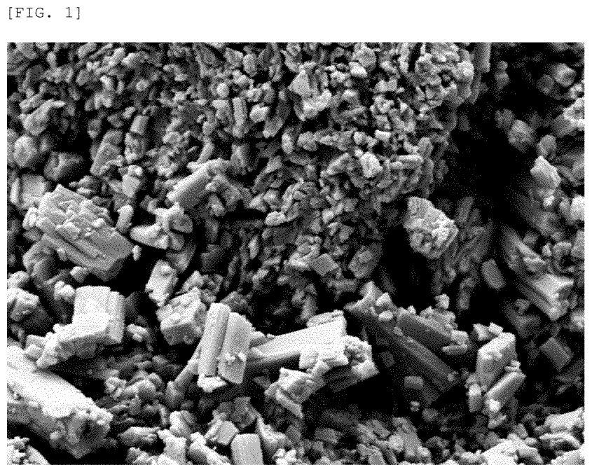

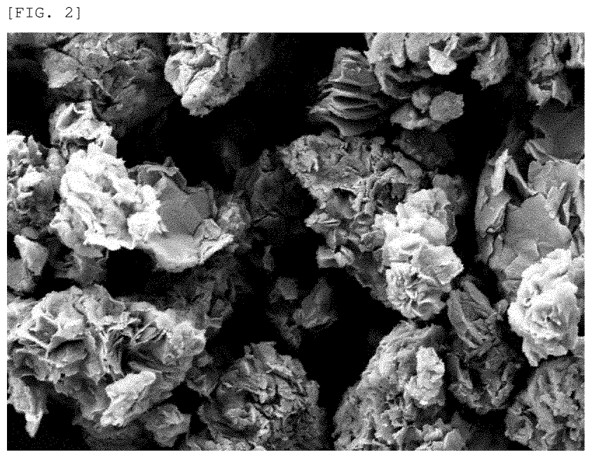

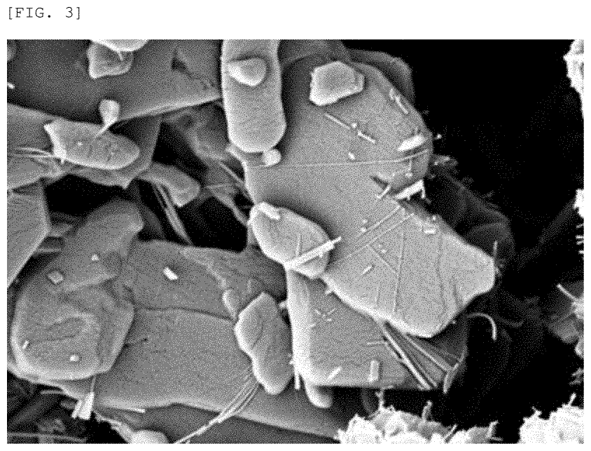

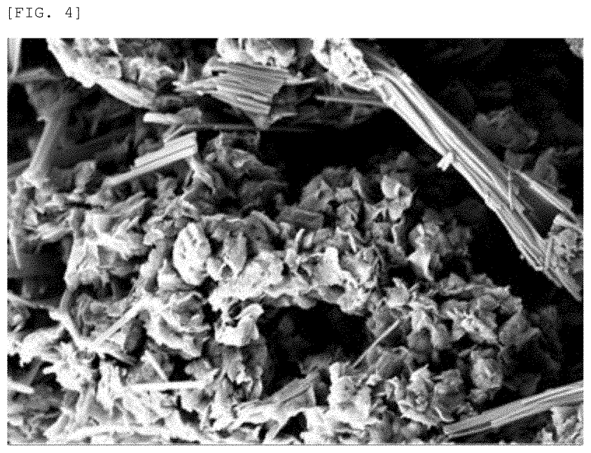

FIG. 1 shows a scanning electron microscopy photograph of a doxazosin compound itself and FIG. 2 shows a scanning electron microscopy photograph of a doxazosin-bentonite complex prepared according to Example 2. FIG. 3 shows a scanning electron microscopy photograph of a prazosin compound itself and FIG. 4 shows a scanning electron microscopy photograph of a prazosin-bentonite complex prepared according to Example 4.

Referring to FIGS. 1 to 4, it was confirmed that in SEM images for the doxazosin compound itself and the prazosin compound itself, the drug compound was in a crystal state, but in the case of forming the complex with bentonite, the drug compound was in an amorphous state.

### Experimental Example 3

The crystal form of the drug-bentonite complex was observed using X-ray diffraction analysis to confirm the morphological properties of the prepared drug-bentonite complex.

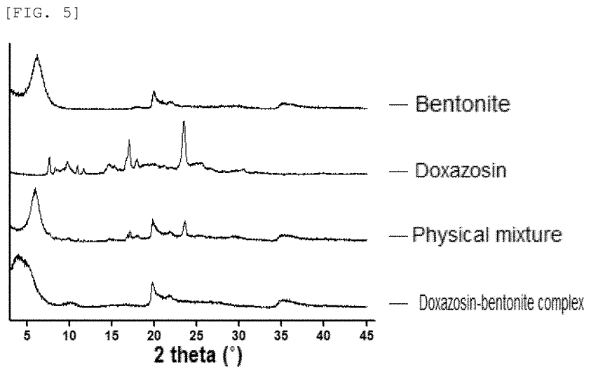

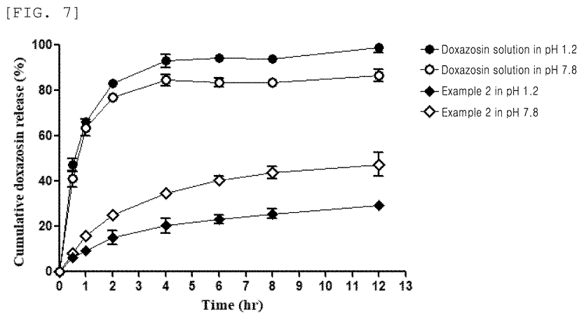

FIG. 5 illustrates X-ray diffraction analysis results of a doxazosin compound (Doxazosin), bentonite powder (Bentonite), a physical mixture of doxazosin and bentonite of Comparative Example 1 (Physical mixture), and a doxazosin-bentonite complex of Example 2 (Doxazosin-bentonite complex) and FIG. 7 illustrates X-ray diffraction analysis results of a prazosin compound (Prazosin), bentonite powder (Bentonite), a physical mixture of prazosin and bentonite of Comparative Example 2 (Physical mixture), and a prazosin-bentonite complex of Example 4 (Prazosin-bentonite complex).

As a result, in X-ray diffraction analysis of the physical mixture of doxazosin and bentonite, main peaks due to a specific crystal structure observed in the doxazosin compound were relatively observed clear, but in X-ray diffraction analysis of the doxazosin-bentonite complex, main peaks by a specific crystal structure observed in the doxazosin compound were almost not shown (FIG. 5). Therefore, it was confirmed that the peaks of the doxazosin-bentonite complex were different from the peaks of the doxazosin compound in X-ray diffraction analysis.

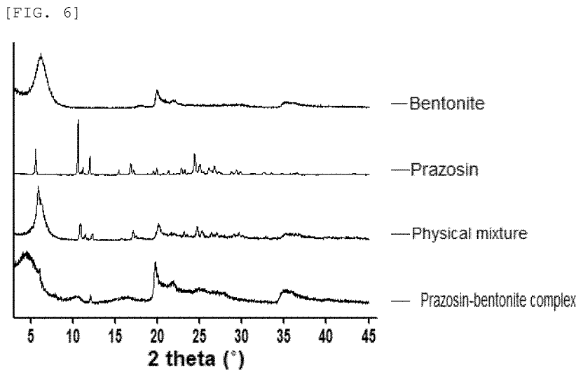

In addition, even in prazosin, similarly to doxazosin, in X-ray diffraction analysis for the prazosin-bentonite complex, main peaks due to the specific crystal structure of the prazosin compound were almost not shown (FIG. 6). Therefore, it was confirmed that the peaks of the prazosin-bentonite complex were different from the peaks of the prazosin compound in X-ray diffraction analysis.

As a result, it was confirmed that when the drug compounds such as doxazosin and prazosin formed the complex with bentonite, the drug compounds and the complex had different peaks in X-ray diffraction analysis, and in the complex, the drug compounds were bound to the layered structure of bentonite and existed in an amorphous state.

### Experimental Example 4

In order to identify the aspect of releasing the drug from the drug-bentonite complex, a drug release experiment using a semi-permeable membrane was conducted. 2 mL of a doxazosin solution dissolved with a doxazosin compound at a concentration of 15 μg/mL and 2 mL of the drug-bentonite complex dispersion solution prepared according to Example 2 were added in a semi-permeable membrane (molecular weight cut-off 12,000 to 14,000 Da) bag and immersed in 28 mL of an eluate, and then the concentration of the drug released from the outside was measured to calculate a cumulative amount of the released drug over time. The amount of the drug-bentonite complex was adjusted so that the drug final concentration inside the elution system became 1 μg/ml. At this time, as the eluate, a 0.1 N hydrochloric acid solution of pH 1.2 and a phosphate buffer of pH 7.8 were used and the elution was performed while maintaining 37° C. While the eluate outside the semi-permeable membrane was partially taken by time and the same amount of eluate was compensated, the experiment was conducted, and the drug concentration of the taken sample was analyzed using high performance liquid chromatography.

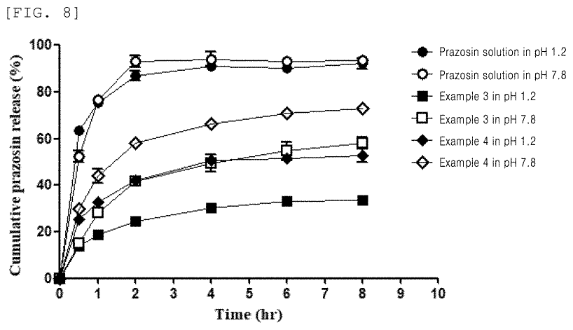

The results were shown in FIGS. 7 and 8. FIG. 7 shows a graph of measuring drug release amounts over time on a doxazosin solution and a doxazosin-bentonite complex dispersion solution prepared according to Example 2 and FIG. 8 shows a graph of measuring drug release amounts over time on a prazosin solution, a prazosin-bentonite complex dispersion solution prepared according to Example 3, and a prazosin-bentonite complex dispersion solution prepared according to Example 4.

In the case of the doxazosin solution, 50% or more of the drug was released within initial 1 hour, and most of doxazosin was released within initial 4 hours. On the other hand, in the doxazosin-bentonite complex dispersion solution, doxazosin was slowly released for about 12 hour or more (FIG. 7). Therefore, as the release test result in the semi-permeable membrane having the molecular weight cut-off of 12,000 to 14,000 Da, the release rate (membrane release amount of drug per unit time) of the doxazosin solution was two times larger than the release rate of doxazosin in the doxazosin-bentonite complex dispersion solution. For example, after 4 hours of the test, the release amount of the doxazosin solution was two times larger than that of doxazosin in the doxazosin-bentonite complex dispersion solution.

In particular, doxazosin showed a fast elution rate in both pH 1.2 and 7.8, while the doxazosin-bentonite complex showed a slow elution rate in both pH 1.2 and 7.8, particularly, showed a significantly low elution rate in pH 1.2. Therefore, it was considered that when doxazosin that may be rapidly dissolved and absorbed in the upper digestive tract was adsorbed onto bentonite, the elution of the doxazosin from the upper digestive tract was inhibited to delay the absorption. It was determined that as the complex moved from the upper digestive tract to the lower digestive tract, the release of the drug was increased by a pH environment and the drug may be continuously released.

In the case of the prazosin solution, 70% or more of the drug was released within initial 1 hour, and most of prazosin was released within initial 4 hours. On the other hand, in the prazosin-bentonite complex dispersion solution, prazosin was slowly released for about 8 hour or more (FIG. 8). Therefore, as the release test result in the semi-permeable membrane having the molecular weight cut-off of 12,000 to 14,000 Da, the release rate (membrane release amount of drug per unit time) of the prazosin solution was larger than the release rate of prazosin in the prazosin-bentonite complex dispersion solution. For example, after 2 hours of the test under a pH 1.2 condition, the release amount of the prazosin solution was two times larger than the release amount of prazosin in the prazosin-bentonite complex dispersion solution. In addition, after 2 hours of the test under a pH 7.8 condition, the release amount of the prazosin solution was significantly larger than the release amount of prazosin in the prazosin-bentonite complex dispersion solution.

In particular, prazosin showed a fast elution rate in both pH 1.2 and 7.8, while the prazosin-bentonite complex showed a slow elution rate in both pH 1.2 and 7.8, particularly, showed a significantly low elution rate in pH 1.2. Therefore, it was considered that when prazosin that may be rapidly dissolved and absorbed in the upper digestive tract was adsorbed onto bentonite, the elution of the prazosin from the upper digestive tract was inhibited to delay the absorption. It was determined that as the complex moved from the upper digestive tract to the lower digestive tract, the release of the drug was increased by a pH environment and the drug may be continuously released.

Accordingly, it can be confirmed that when the drug compound is bound to bentonite to form the complex, the complex can be significantly sustained-released as compared to the drug compound.

### Experimental Example 5

In order to evaluate the pharmacokinetics characteristic of the prepared drug-bentonite complex, after the drug-bentonite complex was orally administered to rats as a subject, the trend of plasma concentration over time was observed. After the femoral region of the fixed rat was shaved, the blood-gathering was prepared by injecting a cannula. A drug-bentonite complex dispersion solution as an experimental group and a drug aqueous solution as a control group were administered using an oral zonde and then a certain amount of plasma was collected through the cannula for each predetermined time and the plasma drug concentration was measured.

In the case of doxazosin, a doxazosin solution (1 mg/kg) was set as a control group and the doxazosin-bentonite complex (3 mg/kg as doxazosin) of Example 2 and the physical mixture (3 mg/kg as doxazosin) of doxazosin and bentonite of Comparative Example 1 were orally administered. In the case of prazosin, a prazosin solution (1 mg/kg) was set as a control group and the prazosin-bentonite complex (5 mg/kg as prazosin) of Example 4 and the physical mixture (5 mg/kg as prazosin) of prazosin and bentonite of Comparative Example 2 were orally administered. The drug concentrations in the plasma samples were analyzed using a mass spectrometer.

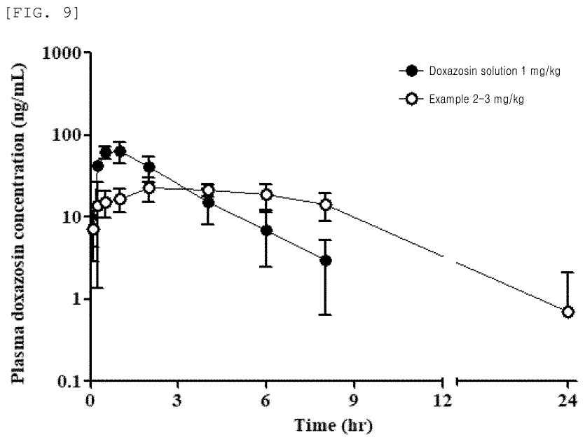

Through a result of analyzing plasma drug concentrations over time after orally administering a doxazosin solution and a doxazosin-bentonite complex dispersion solution as illustrated in FIG. 9, the pharmacokinetics characteristic was examined. In the case of orally administering the doxazosin solution, the plasma concentration reached a maximum plasma concentration (Cmax) within 30 minutes to 1 hour and then was rapidly reduced, while in the case of the bentonite complex preparation, a relatively low maximum plasma concentration (25.2±5.27 ng/mL) was shown, and the plasma concentration of doxazosin was maintained at a constant level for 8 hours without a large change. That is, when the doxazosin solution was applied, high Cmax (maximum plasma concentration) and fast Tmax (time to maximum plasma concentration) were shown, which was shown that the absorption of the drug in the solution state was very fast. On the other hand, when the doxazosin-bentonite complex was applied, the Cmax was lower than that of the doxazosin solution, but Tmax was later than that of the doxazosin solution. That is, when describing pharmacokinetics parameters of doxazosin in Table 3, the time to maximum plasma concentration (Tmax) when orally administering doxazosin-bentonite was 2.56±1.56 minutes, which was about 3.4 times longer than 0.75±0.25 minutes of the time to maximum plasma concentration (Tmax) when orally administering the doxazosin solution. In addition, the maximum plasma concentration (Cmax) when orally administering the doxazosin-bentonite complex was 25.2±5.27 ng/mL, which was significantly lower than 67.3±14.2 ng/mL of the maximum plasma concentration (Cmax) when orally administering the doxazosin solution. Accordingly, it was confirmed that the complex prevented a sudden increase in initial plasma concentration, and it was confirmed that the plasma concentration was maintained for a long time through the sustained-release of doxazosin by adsorbing doxazosin onto bentonite (FIG. 9 and Table 3).

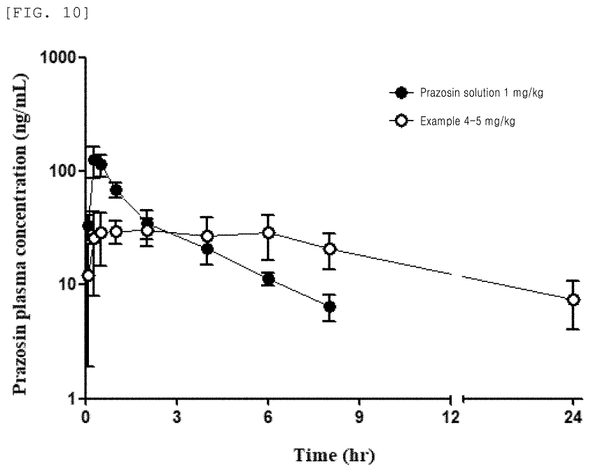

In addition, as a result of analyzing plasma drug concentrations over time after orally administering a prazosin solution and a prazosin-bentonite complex dispersion solution as illustrated in FIG. 10, the pharmacokinetics characteristic was examined. In the case of orally administering 1 mg/kg of the prazosin solution, the plasma concentration reached a maximum plasma concentration within 30 minutes to 1 hour and then was rapidly reduced, while in the case of the bentonite complex preparation, a relatively low maximum plasma concentration (43.0 ng/mL) was shown, and the plasma concentration of prazosin was maintained at a constant level for 8 hours without a large change. That is, when the prazosin solution was applied, high Cmax (maximum plasma concentration) and fast Tmax (time to maximum plasma concentration) were shown, which was shown that the absorption of the drug in the solution state was very fast. On the other hand, when the prazosin-bentonite complex was applied, the Cmax was lower than that of the prazosin solution, but Tmax was later than that of the prazosin solution. That is, when describing pharmacokinetics parameters of prazosin in Table 4, the time to maximum plasma concentration (Tmax) when orally administering prazosin-bentonite was 4.05±2.10 minutes, which was about 10 times longer than 0.38±0.13 minutes of the time to maximum plasma concentration (Tmax) when orally administering the prazosin solution. In addition, the maximum plasma concentration (Cmax) when orally administering the prazosin-bentonite complex was 43.0±4.75 ng/mL, which was significantly lower than 129±36.2 ng/mL of the maximum plasma concentration (Cmax) when orally administering the prazosin solution. Accordingly, it was confirmed that the complex prevented a sudden increase in initial plasma concentration, and it was confirmed that the plasma concentration was maintained for a long time through the sustained-release of prazosin by adsorbing prazosin onto bentonite (FIG. 10 and Table 4).

TABLE 3

| Pharmacokinetics | | |
|---|---|---|
| parameter | Doxazosin | Example 2 |
| Drug dose(Dose, mg/kg) | 1 | 3 |
| Cₘₐₓ (ng/mL) | 67.3 ± 14.2 | 25.2 ± 5.27 |
| Tₘₐₓ (min) | 0.75 ± 0.25 | 2.56 ± 1.56 |
| AUCₗₐₛₜ (ng · min/mL) | 186 ± 54.7 | 195 ± 39 |
| Relative bioavailability (%) | 100 | 34.9 |

TABLE 4

| Pharmacokinetics | | |
|---|---|---|
| parameter | Prazosin | Example 4 |
| Drug dose(Dose, mg/kg) | 1 | 5 |
| Cₘₐₓ (ng/mL) | 129 ± 36.2 | 43.0 ± 4.75 |
| Tₘₐₓ (min) | 0.38 ± 0.13 | 4.05 ± 2.10 |
| AUCₗₐₛₜ (ng · min/mL) | 247 ± 45.1 | 408.9 ± 87.9 |
| Relative bioavailability (%) | 100 | 33.0 |

Through these results, it can be confirmed that when the complex is formed by binding the drug compound to bentonite and orally administered, the drug release is delayed by bentonite and a lower plasma concentration than the drug aqueous solution may be constantly maintained for a long time.

The present disclosure has been described with reference to the preferred embodiments of the present disclosure, but those skilled in the art will understand that the present disclosure can be variously modified and changed without departing from the spirit and the scope of the present disclosure which are defined in the appended claims.

### INDUSTRIAL AVAILABILITY

A controlled-release composition for oral administration of the present disclosure is orally administered to a subject to control the release of a hydrophilic alpha adrenergic blocker compound in the composition, thereby stably and continuously maintaining the plasma drug concentration without a sudden change in the plasma drug concentration.

1. A controlled-release composition for oral administration comprising: a complex of a hydrophilic alpha adrenergic blocker compound or its salt; and a clay mineral as a plate-like carrier with interlayer expandability,
wherein the release of the hydrophilic alpha adrenergic blocker compound in the composition is controlled.

2. The composition of claim 1, wherein the hydrophilic alpha adrenergic blocker compound is prazosin, doxazosin, terazosin, alfuzosin or its pharmaceutically acceptable salt.

3. The composition of claim 1, wherein a time to maximum plasma concentration (tmax) in the oral administration of the composition is 1.5 to 13 times longer than a time to maximum plasma concentration (tmax) in the oral administration of the hydrophilic alpha adrenergic blocker compound aqueous solution.

4. The composition of claim 1, wherein the time to maximum plasma concentration (tmax) in the oral administration of the composition is longer than the time to maximum plasma concentration (tmax) in the oral administration of the hydrophilic alpha adrenergic blocker compound aqueous solution.

5. The composition of claim 1, wherein a maximum plasma concentration (Cmax) in the oral administration of the composition is 15% to 80% of a maximum plasma concentration (Cmax) in the oral administration of the hydrophilic alpha adrenergic blocker compound aqueous solution.

6. The composition of claim 1, wherein the maximum plasma concentration (Cmax) in the oral administration of the composition is lower than the maximum plasma concentration (Cmax) in the oral administration of the hydrophilic alpha adrenergic blocker compound aqueous solution.

7. The composition of claim 1, wherein an elution rate when adding the complex to an eluate of pH 1.2 is slower than the elution rate when adding the hydrophilic alpha adrenergic blocker compound to the eluate of pH 1.2.

8. The composition of claim 1, wherein an elution rate when adding the complex to an eluate of pH 7.8 is slower than the elution rate when adding the hydrophilic alpha adrenergic blocker compound to the eluate of pH 7.8.

9. The composition of claim 1, wherein the composition is a pharmaceutical composition for preventing or treating hypertension or prostate hypertrophy.

10. The composition of claim 1, wherein the composition is a food composition for preventing or alleviating hypertension or prostate hypertrophy.

11. A method for preparing a controlled-release composition for oral administration comprising preparing a complex by mixing a drug aqueous solution prepared by dissolving an alpha adrenergic blocker compound or its pharmaceutically acceptable salt in an acidic aqueous solvent; and a clay mineral suspension as a plate-like carrier with interlayer expandability to adsorb the compound onto the clay mineral.

12. The preparing method of claim 11, wherein the drug aqueous solution has pH of more than 0 to less than 7.
